(19) United States
(12) Patent Application Publication
(10) Pub. No.: US 2026/0191492 A1
(43) Pub. Date: Jul. 9, 2026

(54) X-RAY IMAGING APPARATUS AND METHOD

(71) Applicants: VATECH Co., Ltd., Gyeonggi-do (KR); VATECH EWOO Holdings Co., Ltd., Gyeonggi-do (KR)

(72) Inventor: Chul Kyu PARK, Gyeonggi-do (KR)

(73) Assignees: VATECH Co., Ltd., Gyeonggi-do (KR); VATECH EWOO Holdings Co., Ltd., Gyeonggi-do (KR)

(21) Appl. No.: 19/558,424

(22) Filed: Mar. 5, 2026

(30) Foreign Application Priority Data
Mar. 18, 2025 (KR) 10-2025-0034581

(51) Int. Cl. A61B 6/00; A61B 6/03; A61B 6/51

(52) U.S. Cl. CPC A61B 6/5247; A61B 6/03; A61B 6/51; A61B 6/5205

(57) ABSTRACT

Proposed is an X-ray imaging apparatus. The X-ray imaging apparatus may include a virtual image generator and an enhanced image generator. The virtual image generator is configured to generate, on the basis of a 3D surface image of an object consisting of multiple tissues, virtual X-ray image information assuming the object consists of only a first tissue. The enhanced image generator is configured to generate enhanced X-ray image information in which a second tissue different from the first tissue is enhanced, by using the virtual X-ray image information and actual X-ray image information on the object.

### CROSS REFERENCE TO RELATED APPLICATION

The present application claims priority to Korean Patent Application No. 10-2025-0034581, filed 18 Mar. 2025, the entire contents of which is incorporated herein for all purposes by this reference.

### BACKGROUND OF THE INVENTION

### Field of the Invention

The present disclosure relates to an X-ray imaging apparatus and method.

### Description of the Related Art

Dual-energy computed tomography (DECT) is a technology for analyzing characteristics of tissue or material by using X-rays of two different energy levels.

In this regard, the X-rays of two energy levels may include a low-energy X-ray and a high-energy X-ray. The low-energy X-ray has relatively high attenuation by a tissue due to a low energy level and is advantageous for identifying a detailed structure of soft tissue. The high-energy X-ray has relatively low attenuation by tissue due to a high energy level and is suitable for enhancing a high-density structure of hard tissue (bones and teeth).

DECT may generate an enhanced image in which soft tissue or hard tissue is enhanced, by processing X-ray images of two energy levels using X-ray attenuation characteristics different for each material.

However, this requires two radiographies using X-rays of two energy levels, which causes a problem of increasing radiation exposure and increasing radiation exposure of an object. In addition, precise matching is required during radiography for two X-ray images, but even slight movement of a patient causes misalignment between the two images, leading to distortions of results.

The technology behind the present disclosure is disclosed in Korean Patent No. 10-1731589.

The foregoing is intended merely to aid in the understanding of the background of the present disclosure, and is not intended to mean that the present disclosure falls within the purview of the related art that is already known to those skilled in the art.

### SUMMARY OF THE INVENTION

The present disclosure is directed to providing an X-ray imaging apparatus and method being capable of generating an image in which a particular tissue is enhanced through only a single X-ray radiography, by using a virtual X-ray image assumed to be filled with soft tissue or hard tissue.

In addition, the present disclosure is directed to providing an X-ray imaging apparatus and method being capable of reducing noise caused by a difference in attenuation levels between tissues by using a virtual X-ray image assumed to be filled with soft tissue or hard tissue, compared to the conventional technique.

However, technical objectives that the embodiment of the present disclosure is intended to achieve are not limited to the above-described technical objectives, and there may be other technical objectives.

According to an embodiment of the present disclosure, there is provided an X-ray imaging apparatus including: a virtual image generator configured to generate, on the basis of a 3D surface image of an object consisting of multiple tissues, virtual X-ray image information assuming the object consists of only a first tissue; and an enhanced image generator configured to generate enhanced X-ray image information enhancing a second tissue different from the first tissue, by using the virtual X-ray image information and actual X-ray image information on the object.

According to an embodiment of the present disclosure, there is provided an X-ray imaging method including: generating, on the basis of a 3D surface image of an object consisting of multiple tissues, virtual X-ray image information assuming the object consists of only a first tissue; and generating enhanced X-ray image information enhancing a second tissue different from the first tissue, by using the virtual X-ray image information and actual X-ray image information on the object.

According to the above-mentioned solutions of the present disclosure, by using a virtual X-ray image assumed to be filled with soft tissue or hard tissue, an image in which a particular tissue is enhanced can be generated through only a single X-ray radiography.

According to the above-mentioned solutions of the present disclosure, by using a virtual X-ray image assumed to be filled with soft tissue or hard tissue, noise caused by a difference in attenuation levels between tissues can be reduced, compared to the conventional technique.

However, effects achieved by the present disclosure are not limited to the above-described effects, and there may be other effects.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1 is a schematic block diagram illustrating an X-ray imaging apparatus according to an embodiment of the present disclosure;

FIG. 2 is a diagram illustrating a 3D surface image according to an embodiment of the present disclosure;

FIG. 3 is a diagram illustrating an original panoramic image as actual X-ray image information according to an embodiment of the present disclosure;

FIG. 4 is a diagram illustrating an enhanced panoramic image as enhanced X-ray image information according to an embodiment of the present disclosure; and

FIG. 5 is an operation flowchart illustrating an X-ray imaging method according to an embodiment of the present disclosure.

### DETAILED DESCRIPTION OF THE INVENTION

The present disclosure relates to an X-ray imaging apparatus and method.

Throughout the present specification, a subject may mean a target, that is, a patient, who undergoes diagnosis and observation regarding teeth (oral cavity) using a medical image display device according to an embodiment of the present disclosure. An object may refer to a radiography portion or a radiography region of the subject. A user may refer to a person, that is, a medical staff (doctor, nurse, etc.), who performs diagnosis and observation regarding the teeth (oral cavity) of the object using the medical image display device according to an embodiment of the present disclosure. However, no limitation thereto is imposed.

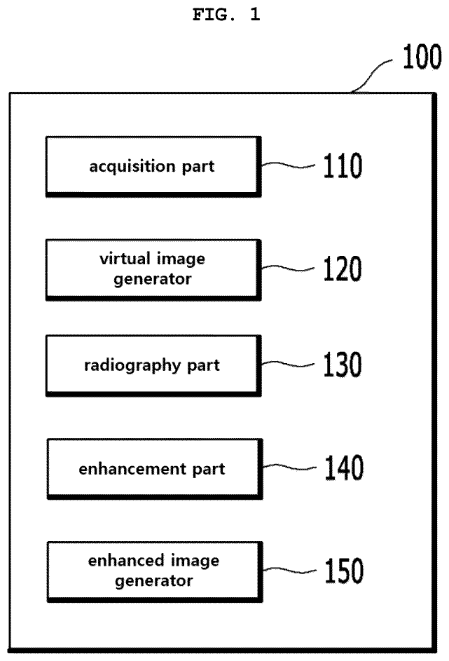

FIG. 1 is a schematic block diagram illustrating an X-ray imaging apparatus according to an embodiment of the present disclosure.

Referring to FIG. 1, an X-ray imaging apparatus 100 (hereinafter, referred to as the apparatus 100) may include an acquisition part 110, a virtual image generator 120, a radiography part 130, an enhancement part 140, and an enhanced image generator 150.

In addition, although not shown, the apparatus 100 may further include a display, an input part, a controller, and a storage.

For reference, the display may refer to a display screen, and may be configured to output a 3D surface image, an actual X-ray image, a virtual X-ray image, and an enhanced image according to an embodiment of the present disclosure. For example, the display may refer to at least one of various display devices capable of displaying images including all types of wireless communication devices, such as a smartphone, a smart pad, a tablet PC, a wearable device, and terminals for a Personal Communication System (PCS), the Global System for Mobile Communications (GSM), Personal Digital Cellular (PDC), Personal Handy-phone System (PHS), Personal Digital Assistant (PDA), International Mobile Telecommunication (IMT)-2000, Code-Division Multiple Access (CDMA)-2000, Wideband Code-Division Multiple Access (WCDMA), and a wireless broadband Internet (Wibro), and fixed terminals such as a desktop computer and a smart TV, and an LCD display, an LED display, an AMOLED display, and a CRT display, but is not limited thereto.

For reference, the input part may be configured to receive a user input for selection and management of an image to be displayed through the display. Examples of the input part may include a mouse, a keyboard of a computer, a keypad, and a touchpad, but are not limited thereto. The input part may include a graphic user interface controllable using the above-described input part.

In addition, the controller may refer to a central processing unit that controls the overall operation of the apparatus 100 according to an embodiment of the present disclosure. For example, the controller may be implemented using at least one of application-specific integrated circuits (ASICs), digital signal processors (DSPs), digital signal processing devices (DSPDs), programmable logic devices (PLDs), field-programmable gate arrays (FPGAs), processors, controllers, micro-controllers, and microprocessors, or may be implemented as a firmware/software module executable on the above-described hardware platform. Herein, the firmware/software module may be implemented by one or more software applications written in an appropriate program language.

In addition, the storage may be configured to store various images, variable values, a 3D surface image and 3D surface data therefor, an actual X-ray image and at least one piece of original projection data therefor, a virtual X-ray image and at least one piece of virtual projection data therefor, and an enhanced image and at least one piece of enhanced projection data therefor, for controlling the operation of the apparatus (100) according to an embodiment of the present disclosure. For example, the storage may be implemented as any one storage medium of the following: a flash memory type, a hard disk type, a MultiMedia Card (MMC), a card-type memory (e.g., a Secure Digital (SD) card or eXtreme Digital (xD) card), random-access memory (RAM), static random-access memory (SRAM), read-only memory (ROM), electrically erasable programmable read-only memory (EEPROM), a programmable read-only memory (PROM), a magnetic memory, a magnetic disk, and an optical disk, but is not limited thereto.

Herein, according to an embodiment of the present disclosure, the virtual image generator 120, the enhancement part 130, and the enhanced image generator 140 may be embedded in the controller or the storage or both described above.

In addition, the input part, the controller, the storage, and the display described above may be interconnected via a network. Examples of the network may include a 3^{rd} Generation Partnership Project (3GPP) network, a long-term evolution (LTE) network, a 5G network, a World Interoperability for Microwave Access (WIMAX) network, wired/wireless Internet, a local area network (LAN), a wireless local area network (Wireless LAN), a wide area network (WAN), a personal area network (PAN), a Bluetooth network, a Wi-Fi network, a near-field communication (NFC) network, a satellite broadcasting network, an analog broadcasting network, and a digital multimedia broadcasting (DMB) network, but are not limited thereto.

Hereinafter, an X-ray imaging process according to an embodiment of the present disclosure will be described in detail.

Hereinafter, X-ray image information including virtual X-ray image information, actual X-ray image information, and enhanced X-ray image information may be, for example, a cephalometric X-ray image reconstructed using one or more pieces of projection information (including virtual projection information, original projection information, and enhanced projection information) in the dental field, or a CT image or panoramic image reconstructed using a plurality of pieces of projection information, but is not limited thereto. In addition, the virtual projection information and the original projection information may be acquired by radiographing the object at each of a plurality of angles around the object using a virtual or actual X-ray irradiator and a virtual or actual X-ray detector. Accordingly, the virtual X-ray image information and the actual X-ray image information may include all types of image information reconstructed using the virtual projection information and the original projection information, respectively.

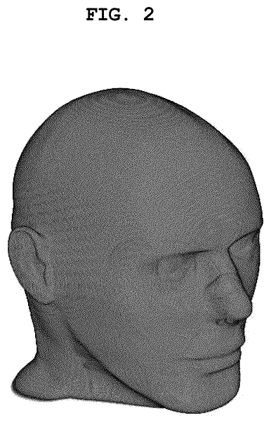

FIG. 2 is a diagram illustrating a 3D surface image according to an embodiment of the present disclosure.

Referring to FIG. 2, the acquisition part 110 may acquire a 3D surface image of the object. Herein, the object may be the subject's head and may consist of multiple tissues. In addition, the tissues may include hard tissue, such as bones or teeth, having a relatively high X-ray attenuation rate, soft tissue, such as skin, having a relatively low X-ray attenuation rate, and various other tissues having an X-ray attenuation rate between those of the hard tissue and the soft tissue.

In addition, the acquisition part 110 may include a 3D camera based on an optical scanner. The 3D surface image may be acquired using a 3D camera, or may be acquired from 3D surface information on the head, which is the radiography portion of the subject. In addition, the 3D surface image may be in a state in which the inside is empty, unlike the object that consists of multiple tissues.

According to an embodiment of the present disclosure, the virtual image generator 120 may generate the virtual X-ray image information for a case in which the object consists of only a first tissue, on the basis of the 3D surface image. In other words, the virtual image generator 120 may assume that the inside of the 3D surface is filled with only the first tissue, which is any one of a plurality of tissues, and generate the virtual X-ray image information as a result of virtual X-rays passing through the 3D surface image assumed to be filled with only the first tissue. That is, the virtual X-ray image information may be generated based on the assumption that the object are irradiated by virtual X-rays. Herein, the first tissue may be any one of tissues (hard tissue, soft tissue, and other tissues) forming the head.

Herein, the virtual X-ray image information may be reconstructed from a plurality of pieces of virtual projection information acquired at different angles with respect to the object. In this regard, the virtual X-ray projection information may be acquired at a plurality of different angles around the object by virtual X-rays.

According to an embodiment of the present disclosure, the radiography part 130 may acquire the actual X-ray image information on the object. The radiography part 130 may include an X-ray irradiator that irradiates the object with X-rays, and an X-ray detector that detects X-rays passing through the object. In other words, the radiography part 130 may generate the actual X-ray image information on the object on the basis of a detection result detected by the X-ray detector by irradiating the object with actual X-rays. Specifically, the detection result may include a plurality of pieces of original projection information acquired at different angles with respect to the object by the X-ray detector, and the actual X-ray image information may be reconstructed from the plurality of pieces of original projection information. However, the present disclosure is not limited thereto, and the actual X-ray image information may be transmitted from a separate X-ray radiography apparatus outside the X-ray imaging apparatus 100.

Herein, the virtual X-ray projection information of the virtual X-ray image information and the original projection information of the actual X-ray image information may correspond to each other in a one-to-one manner. In other words, the virtual X-ray projection information of the virtual X-ray image information and the original projection information of the actual X-ray image information corresponding to each other may be X-ray projection information obtained at the same angle with respect to the object.

In this regard, the 3D surface image may be acquired during radiography of the actual X-ray image information by the radiography part 130. For example, in the case of a dental X-ray image apparatus, for panoramic or CT radiography, an X-ray irradiator and an X-ray detector rotate while facing each other with an object interposed therebetween to acquire a plurality of pieces of projection data at a plurality of angles around the object, and the acquired plurality of pieces of projection data are used to reconstruct a panoramic or CT image. Accordingly, when a separate 3D camera is provided at the X-ray irradiator and the X-ray detector, during acquisition of the plurality of pieces of projection data through rotation of the X-ray irradiator and the X-ray detector facing each other, the 3D camera may acquire a plurality of images at a plurality of angles with respect to the object, whereby the 3D surface image may be radiographed and acquired.

According to an embodiment of the present disclosure, the enhancement part 140 may adjust any one image such that any one tissue (the first tissue) shown in any one image information among the virtual X-ray image information and the actual X-ray image information on the object corresponds to the first tissue shown in the other image information among the virtual X-ray image information and the actual X-ray image information. Herein, adjustment may be globally filtering any one piece of image information so that a data value for the first tissue shown in the one piece of image information becomes equal to a data value for the first tissue shown in the other one piece of image information, such that the first tissue in the corresponding image information is enhanced.

For example, when a tissue filling the inside in the virtual X-ray image information is hard tissue, the virtual X-ray image information is image information in which no other tissues except for the hard tissue are displayed, so only a data value (e.g., a Hounsfield Unit (HU) value indicating brightness according to density of material) for the hard tissue is displayed. Herein, the enhancement part 140 may adjust a data value for the hard tissue including bones and teeth displayed in the actual X-ray image information to a level equal to a data value in the virtual X-ray image information.

As another example, when a tissue filling the inside in the virtual X-ray image information is soft tissue, the virtual X-ray image information is image information in which no other tissues except for the soft tissue are displayed, so only a data value for the soft tissue is displayed. Herein, the enhancement part 140 may adjust a data value for the soft tissue, such as muscle, fat, blood vessels, and nerves, displayed in the actual X-ray image information to a level equal to a data value in the virtual X-ray image information.

According to an embodiment of the present disclosure, the enhanced image generator 150 may generate the enhanced X-ray image information in which a second tissue different from the first tissue is enhanced, using the virtual X-ray image information and the actual X-ray image information. In this regard, the first tissue and the second tissue may have different X-ray attenuation coefficients, and the enhanced image generator 150 may generate the enhanced X-ray image information using the difference between the respective X-ray attenuation coefficients of the first tissue and the second tissue.

In other words, the enhanced image generator 150 may generate the enhanced image in which any one tissue (e.g., the first tissue) is excluded and another tissue (e.g., the second tissue) is enhanced, by subtracting the virtual X-ray image information from the actual X-ray image information. For example, assuming a case in which the actual X-ray image information consists of the first tissue and the second tissue, when the virtual X-ray image information is an image filled with the first tissue (or the second tissue), the enhanced image generator 150 may generate the enhanced X-ray image information in which the second tissue (or the first tissue) is enhanced by subtracting the virtual X-ray image information from the actual X-ray image information, whereby the first tissue (or the second tissue) is excluded from the actual X-ray image and only the second tissue (or the first tissue) remains.

In other words, when a tissue filling the inside in the virtual X-ray image information is hard tissue, the enhanced image generator 150 may generate the enhanced X-ray image information in which soft tissue is enhanced by subtracting the virtual X-ray image information from the actual X-ray image information in which the hard tissue is enhanced by the enhancement part 140, whereby the hard tissue is excluded from the actual X-ray image information and only the soft tissue remains. In addition, when a tissue filling the inside in the virtual X-ray image information is soft tissue, the enhanced image generator 140 may generate the enhanced X-ray image information in which hard tissue is enhanced by subtracting the virtual X-ray image information from the actual X-ray image information in which the soft tissue is enhanced by the enhancement part 140, whereby the soft tissue is exclude from the actual X-ray image information and only the hard tissue remains.

In this regard, for convenience of description, the above description has been made using the virtual X-ray image information, the actual X-ray image information, and the enhanced X-ray image information, but the enhanced image generator 150 may be configured to perform an operation between the virtual X-ray image information and the actual X-ray image information on a projection information basis in order to generate an image in which a particular tissue is enhanced. That is, the enhanced X-ray image information may be reconstructed from a plurality of pieces of enhanced projection information acquired on the basis of respective pieces of projection information acquired at angles corresponding to each other among a plurality of pieces of virtual projection information and a plurality of pieces of original projection information.

Specifically, the enhanced image generator 150 may acquire a plurality of pieces of enhanced projection information by respectively subtracting each of the plurality of pieces of virtual projection information from a corresponding one of the plurality of pieces of original projection information in a one-to-one manner, and generate the enhanced X-ray image information by reconstructing the plurality of pieces of enhanced projection information. As described above, the enhanced image generator 150 generates the enhanced X-ray image information by subtracting the virtual X-ray image information from the actual X-ray image information. Specifically, each of the plurality of pieces of virtual projection information for the virtual X-ray image information may be subtracted from each of the plurality of pieces of original projection information for the actual X-ray image information in a one-to-one manner. In addition, through this, the enhanced image generator 150 may acquire a plurality of pieces of projection information, that is, a plurality of pieces of enhanced projection information, in each of which any one tissue is enhanced.

For reference, although it has been described above that a case in which a particular tissue is excluded and only another tissue remains by subtracting virtual X-ray image information from actual X-ray image information is “enhanced”, the actual X-ray image information may include various tissues in addition to soft tissue and hard tissue and even the same tissue may have different data values due to superimposition. Accordingly, the enhanced X-ray image information acquired by subtraction between the two types of X-ray image information may include a case in which a particular tissue is shown relatively weakly. In other words, the enhanced X-ray image information may include all types of image information in which a particular tissue is removed or shown weakly and another tissue is thus shown relatively prominently.

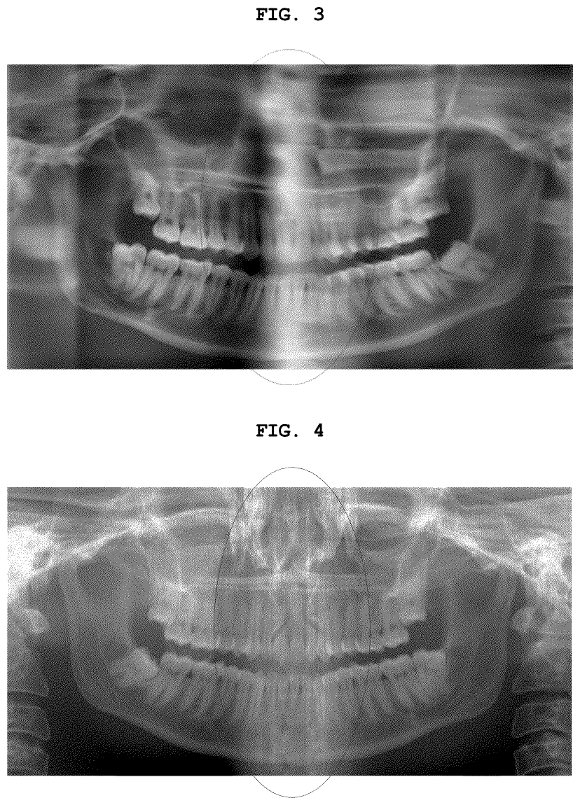

FIG. 3 is a diagram illustrating an original panoramic image as actual X-ray image information according to an embodiment of the present disclosure. In addition, FIG. 4 is a diagram illustrating an enhanced panoramic image as enhanced X-ray image information according to an embodiment of the present disclosure.

Referring to FIG. 3, it can be seen that in the original panoramic image, both soft tissue and hard tissue are displayed in the image, so it is difficult to identify detailed shapes and characteristics of any one tissue.

On the other hand, referring to FIG. 4, it can be seen that in the enhanced panoramic image, any one tissue is displayed in an enhanced manner. Specifically, FIG. 4 shows an image in which hard tissue is enhanced, and may display a cervical vertebrae region indicated by a ellipse in more detail, compared to the original panoramic image shown in FIG. 3.

Accordingly, the X-ray imaging apparatus 100 may generate an enhanced image in which a particular tissue is enhanced, and unlike the conventional DECT requiring two radiographies, the X-ray imaging apparatus 100 requires only a single radiography, thereby reducing a radiation dose compared to the conventional technique.

In addition, as described above, the X-ray imaging apparatus 100 may generate an image in which a particular tissue is enhanced by using a virtual X-ray image assumed to be filled with soft tissue or hard tissue, through only a single X-ray radiography. In addition, by using a virtual X-ray image assumed to be filled with soft tissue or hard tissue, the X-ray imaging apparatus 100 may reduce noise caused by a difference in attenuation levels between tissues, compared to the conventional technique.

Hereinafter, based on the details described above, the operation flow of the present disclosure will be simply described.

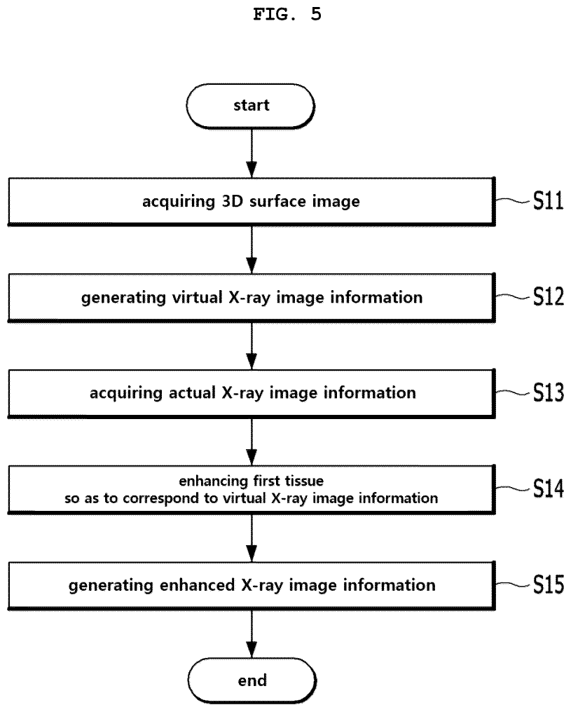

FIG. 5 is an operation flowchart illustrating an X-ray imaging method according to an embodiment of the present disclosure.

The X-ray imaging method shown in FIG. 5 may be performed by the X-ray imaging apparatus 100 described above. Accordingly, even if a particular description is omitted below, the description provided for the X-ray imaging apparatus 100 may be equally applicable to the description of the enhanced image generation method.

Referring to FIG. 5, in step S11, the acquisition part 110 including the 3D camera may acquire the 3D surface image of the object.

Next, in step S12, the virtual image generator 120 may generate, on the basis of the 3D surface image of the object consisting of multiple tissues, virtual X-ray image information for a case in which the object consists of only the first tissue. Herein, the virtual X-ray image information may be reconstructed from a plurality of pieces of virtual projection information acquired at different angles with respect to the object.

Next, in step S13, the radiography part 130, including the X-ray irradiator for irradiating the object with X-rays and the X-ray detector for detecting X-rays passing through the object, may acquire actual X-ray image information on the object. Herein, the actual X-ray image information may be reconstructed from a plurality of pieces of original projection information acquired at different angles with respect to the object.

Next, in step S14, the enhancement part 140 may adjust any one image such that the first tissue shown in any one image information among the virtual X-ray image information and the actual X-ray image information on the object corresponds to the first tissue shown in the other image information among the virtual X-ray image information and the actual X-ray image information. Herein, the actual X-ray image information may be reconstructed from a plurality of pieces of original projection information acquired at different angles with respect to the object. Herein, the first tissue and the second tissue may have different X-ray attenuation coefficients.

Next, in step S15, the enhanced image generator 150 may generate the enhanced X-ray image information in which the second tissue different from the first tissue is enhanced, using the virtual X-ray image information and the actual X-ray image information on the object from the actual X-ray image. Specifically, in step S15, the enhanced image generator 150 may generate the enhanced X-ray image information using a difference between the respective X-ray attenuation coefficients of the first tissue and the second tissue. Herein, the enhanced X-ray image information may be reconstructed from a plurality of pieces of enhanced projection information acquired on the basis of respective pieces of projection information acquired at angles corresponding to each other among a plurality of pieces of virtual projection information and a plurality of pieces of original projection information. In addition, the enhanced X-ray image information may be X-ray panoramic image information or CT image information.

In the above description, steps S11 to S15 may be further divided into additional steps or combined into fewer steps according to an embodiment of the present disclosure. In addition, some steps may be omitted when necessary, and the order of the steps may be changed.

An X-ray imaging method according to an embodiment of the present disclosure may be implemented as program instructions executable by various computer means and may be recorded on a computer-readable recording medium. The computer-readable recording medium may include program instructions, data files, data structures, and the like separately or in combinations. The program instructions to be recorded on the computer-readable recording medium may be specially designed and configured for embodiments of the present disclosure or may be well-known to and be usable by those skilled in the art of computer software. Examples of the computer-readable recording medium include magnetic recording media such as hard disks, floppy disks and magnetic tapes; optical data storage media such as CD-ROMs or DVD-ROMs; magneto-optical media such as floptical disks; and hardware devices, such as read-only memory (ROM), random-access memory (RAM), and flash memory, which are particularly structured to store and implement the program instructions. Examples of the program instructions include not only a mechanical language code formatted by a compiler but also a high level language code that may be implemented by a computer using an interpreter, and the like. The hardware devices may be configured to be operated by one or more software modules or vice versa to conduct the operation according to the present disclosure.

In addition, the X-ray imaging method described above may also be implemented in the form of a computer program or an application executed by a computer and stored on a recording medium.

Although a preferred embodiment of the present disclosure has been described for illustrative purposes, those skilled in the art will appreciate that various modifications, additions and substitutions are possible, without departing from the scope and spirit of the disclosure as disclosed in the accompanying claims.

What is claimed is:

1. An X-ray imaging apparatus, comprising:
a virtual image generator configured to generate, on the basis of a 3D surface image of an object consisting of multiple tissues, virtual X-ray image information assuming the object consists of only a first tissue; and
an enhanced image generator configured to generate enhanced X-ray image information enhancing a second tissue different from the first tissue, by using the virtual X-ray image information and actual X-ray image information on the object.

2. The X-ray imaging apparatus of claim 1, wherein the virtual X-ray image information is reconstructed from a plurality of pieces of virtual projection information acquired at different angles with respect to the object, and
the actual X-ray image information is reconstructed from a plurality of pieces of original projection information acquired at the different angles with respect to the object, and
the enhanced X-ray image information is reconstructed from a plurality of pieces of enhanced projection information acquired on the basis of respective pieces of the projection information acquired at the angles corresponding to each other among the plurality of pieces of virtual projection information and the plurality of pieces of original projection information.

3. The X-ray imaging apparatus of claim 2, wherein the enhanced X-ray image information is X-ray panoramic image information or CT image information.

4. The X-ray imaging apparatus of claim 1, wherein the first tissue and the second tissue have different X-ray attenuation coefficients, and
the enhanced image generator is configured to generate the enhanced X-ray image information using a difference between the respective X-ray attenuation coefficients of the first tissue and the second tissue.

5. The X-ray imaging apparatus of claim 1, further comprising:
an acquisition part including a 3D camera, and configured to acquire the 3D surface image of the object; and
a radiography part including an X-ray irradiator configured to irradiate the object with X-rays and an X-ray detector configured to detect the X-rays passing through the object, the radiography part being configured to acquire the actual X-ray image information on the object.

6. The X-ray imaging apparatus of claim 1, wherein the virtual X-ray image information is generated on the basis of an assumption that the object are irradiated by virtual X-rays, and
the actual X-ray image information is radiographed by irradiating the object with actual X-rays.

7. An X-ray imaging method, comprising:
generating, on the basis of a 3D surface image of an object consisting of multiple tissues, virtual X-ray image information assuming the object consists of only a first tissue; and
generating enhanced X-ray image information enhancing a second tissue different from the first tissue, by using the virtual X-ray image information and actual X-ray image information on the object.
